(19) United States
(12) Patent Application Publication
(10) Pub. No.: US 2007/0293678 A1
(43) Pub. Date: Dec. 20, 2007

(54) Process For The Preparation Of 6, 8-Subs Tituted '1, 7 Naphthpyridin Derivatives By Reacting The 8-Halo-'1, 7 Naphthpyrid In-Derivate With An Organic Boronic Acid Derivatives And Intermadiates Of This Process

(76) Inventors: Xinglong Jiang, Hillsborough, NJ (US); Prasad Koteswara Kapa, Parsippany, NJ (US); George Tien-San Lee, Towaco, NJ (US); Edwin Bernard Villhauer, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/574,255

(22) PCT Filed: Sep. 12, 2005
(86) PCT No.: PCT/US05/32909

§ 371 Date: Feb. 26, 2007

Related U.S. Application Data
(60) Provisional application No. 60/609,576, filed on Sep. 14, 2004.

(51) Int. Cl. C07D 471/04; C07D 213/81

(52) U.S. Cl. 546/122; 546/323

(57) ABSTRACT

A process for the manufacture of isoquinoline and 1,7-naphthyridine derivatives of formula 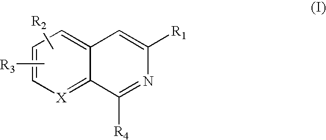
wherein R₁, R₂, R₃, R₄ and X have the meanings as indicated in the specification. The process utilizes readily available starting materials of the formulae 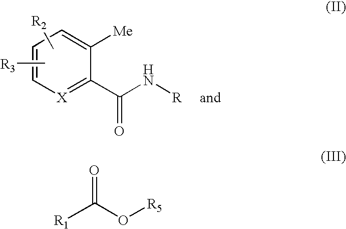
or compounds prepared from such starting materials wherein R₁, R₂, R₃ and X have meanings as defined for formula (I); and R and R₅ are independently C₁-C₇-alkyl.

Inhibition of phosphodiesterase type 4 (PDE4) enzyme represents a promising new approach for the treatment of chronic inflammatory diseases such as asthma, chronic obstructive pulmonary disease (COPD) and rheumatoid arthritis.

The present invention relates to a new process for the manufacture of certain PDE4 inhibitors and intermediates thereof. More specifically, the present invention provides methods for the preparation of isoquinoline and 1,7-naphthyridine derivatives, e.g., those disclosed in international patent application WO 03/039544, U.S. Pat. No. 5,747,506 and U.S. Pat. No. 6,136,821.

Accordingly, the present invention provides a practical and versatile process for the manufacture of compounds of the formula (I)

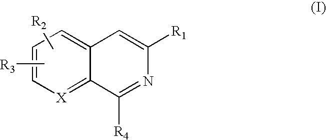

wherein R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,

or R₁ is C₃-C₁₂-cycloalkyl optionally substituted by one or two of C₁-C₇-alkyl, hydroxy, C₁-C₇-alkoxy, C₁-C₇-alkylthio or carboxy,

or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₁ is heteroaryl optionally substituted by C₁-C₇- alkyl, C₁-C₇-alkoxy or halo;

R₂ and R₃ are independently hydrogen or C₁-C20-alkoxy;

R₄ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₄ is heteroaryl optionally substituted by C₁-C₇- alkyl, C₁-C₇-alkoxy or halo; and

X is N or CH;

or a salt thereof;

which process utilizes readily available starting materials of formulae

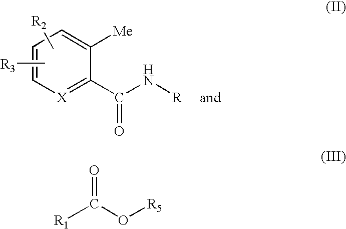

or compounds prepared from such starting materials wherein R₁, R₂, R₃ and X have meanings as defined for formula (I); R and R₅ are independently C₁-C₇-alkyl.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following.

Listed below are definitions of various terms used to describe the compounds of the instant invention. These definitions apply to the terms as they are used throughout the specification unless they are otherwise limited in specific instances either individually or as part of a larger group.

The term “C₁-C₂₀-alkyl” as used herein refers to straight or branched chain hydrocarbon groups having 1 to 20 carbon atoms, for example methyl, ethyl, propyl, isopropyl, n-butyl, t-butyl, isobutyl, pentyl, hexyl, isohexyl, heptyl, 4,4-dimethylpentyl or octyl. Preferably C₁-C₂₀-alkyl is C₁-C₇-alkyl. C₁-C₂₀-alkyl may be substituted by one or two of hydroxy, C₃-C₁₂-cycloalkyl, C₆-C₁₂-aryl, C₁-C₇-alkoxy, thiol, C₁-C₇-alkylthio or carboxy.

The term “C₃-C₁₂-cycloalkyl” as used herein refers to cycloalkyl having 3 to 12 ring carbon atoms. These may be monocyclic, bicyclic or tricyclic hydrocarbon groups. C₃-C₁₂-cycloalkyl” may be substituted by one or two of C₁-C₇-alkyl, hydroxy, C₁-C₇-alkoxy, C₁-C₇-alkylthio or carboxy.

When C₃-C₁₂-cycloalkyl is monocyclic it is preferably cyclopropyl, cyclobutyl, cyclopentyl, cyclopentenyl, cyclohexyl or cyclohexenyl. When C₃-C₁₂-cycloalkyl is bicyclic it is preferably bornyl, indyl, hexahydroindyl, tetrahydro-naphthyl, decahydronaphthyl, bicyclo[2.1.1hexyl, bicyclo[2.2.1]heptyl, bicyclo[2.2.1]-heptenyl, 6,6-dimethylbicyclo[3.1.1]heptyl, 2,6,6-tri-methylbicyclo[3.1.1]heptyl and bicyclo[2.2.2]octyl. When C₃-C₁₂-cycloalkyl is tricyclic it is preferably adamantyl.

C₃-C₁₂-cycloalkyl is especially preferably C₃-C₆-cycloalkyl substituted by C₁-C₄-alkyl, hydroxy, C₁-C₄-alkoxy, C₁-C₄-alkylthio or carboxy.

The term “halogen” or “halo” refers to fluorine, chlorine, bromine and iodine.

The term “C₁-C20-alkoxy” as used herein refers to straight chain or branched alkoxy having 1 to 20 carbon atoms. Preferably C₁-C₂₀-alkoxy is C₁-C₇-alkoxy, especially C₁-C₄-alkoxy.

The term “C₁-C₇-alkylthio” as used herein refers to denotes C₁-C₇-alkyl linked to —S—.

The term “C₂-C₃-alkylene” as used herein refers to a straight chain bridge of 2 or 3 carbon atoms connected by single bonds (e.g., —(CH₂)ₓ— wherein x is 2 or 3). C₂-C₃-alkylene may be substituted by one or two of C₁-C₄-alkyl.

The term “C₆-C₁₂-aryl” as used herein refers to monocyclic or bicyclic aromatic hydrocarbon groups having 6 to 12 carbon atoms in the ring portion, such as phenyl, naphthyl, tetrahydronaphthyl, biphenyl and diphenyl groups. C₆-C₁₂-aryl may be substituted by one, two three or four substituents selected from C₁-C₇-alkyl, halo, hydroxy, C₁-C₇-alkoxy, C₁-C₇-alkylthio or nitro. Preferably C₆-C₁₂-aryl is phenyl substituted by halo.

The term “monocyclic aryl” as used herein refers to phenyl as described under aryl.

The term “heteroaryl” refers to an aromatic heterocycle, such as 5- to 10-membered heterocyclic ring containing at least one ring heteroatom selected from the group consisting of nitrogen, oxygen and sulphur. Heteroaryl is for example monocyclic or bicyclic aryl, such as pyrrolyl, pyrazolyl, imidazolyl, triazolyl, oxazolyl, isoxazolyl, thiazolyl, iso-thiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl or benzofuryl. Heteroaryl may be substituted by C₁-C₇-alkyl, C₁-C₇-alkoxy or halo.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

Compounds of the invention having basic groups, e.g., pyridyl, isoquinolinyl or naphthyridinyl, can be converted into acid addition salts. The acid addition salts may be formed with mineral acids, organic carboxylic acids or organic sulfonic acids, e.g., hydrochloric acid, maleic acid and methanesulfonic acid, respectively.

Similarly, salts formed with bases, e.g., cationic salts, such as alkali and alkaline earth metal salts, such as sodium, lithium, potassium, calcium, magnesium, as well as ammonium salts, such as ammonium, trimethylammonium, diethylammonium, and tris(hydroxymethyl)-methyl-ammonium salts and salts with amino acids, are possible if an acidic group constitutes part of the structure.

In view of the close relationship between the free compounds and the compounds in the form of their salts, whenever a compound is referred to in this context, a corresponding salt is also intended, provided such is possible or appropriate under the circumstances.

The compounds, including their salts, can also be obtained in the form of their hydrates, or include other solvents used for their crystallization.

As described above, the present invention provides a new process for the manufacture of compounds of the formula (I)

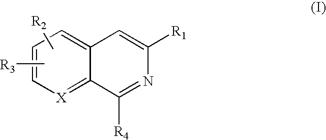

wherein R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,

or R₁ is C₃-C₁₂-cycloalkyl optionally substituted by one or two of C₁-C₇-alkyl, hydroxy, C₁-C₇-alkoxy, C₁-C₇-alkylthio or carboxy,

or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₁ is heteroaryl optionally substituted by C₁-C₇-alkyl, C₁-C₇-alkoxy or halo;

R₂ and R₃ are independently hydrogen or C₁-C₂₀-alkoxy;

R₄ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₄ is heteroaryl optionally substituted by C₁-C₇-alkyl, C₁-C₇-alkoxy or halo; and

X is N or CH;

or a salt thereof.

In a first aspect the process comprises coupling compounds of formula (VI)

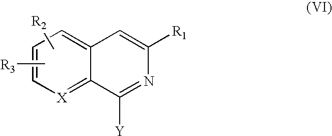

wherein R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,

or R₁ is C₃-C₁₂-cycloalkyl optionally substituted by one or two of C₁-C₇-alkyl, hydroxy, C₁-C₇-alkoxy, C₁-C₇-alkylthio or carboxy,

or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₁ is heteroaryl optionally substituted by C₁-C₇-alkyl, C₁-C₇-alkoxy or halo;

R₂ and R₃ are independently hydrogen or C₁-C₂₀-alkoxy;

X is N or CH;

and Y is chloro or bromo in the presence of a catalyst and a base with a compound of the formula (VII)

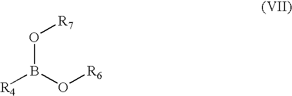

wherein R₄ is C₆-C₁₂-aryl optionally substituted by one, two, three or four substituents selected from C₁-C₇-alkyl, halo, hydroxy, C₁-C₇-alkoxy, C₁-C₇-alkylthio and nitro,

or R₄ is heteroaryl optionally substituted by C₁-C₇- alkyl, C₁-C₇-alkoxy or halo;

and R₆ and R₇ are hydrogen or C₁-C₇-alkyl,

or R₆ and R₇ combined are C₂-C₃ alkylene optionally substituted by one or two of C₁-C₄-alkyl that together with the boron and the oxygen atoms form a 5- or 6-membered ring.

In a second aspect compounds of formula (I) may be prepared by treating compounds of the formula (II)

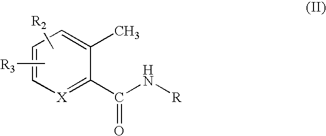

wherein R₂, R₃ and X have meanings as defined above, and R is C₁-C₇-alkyl, preferably t-butyl, with a base such as n-butyllithium, s-butyllithium, t-butyllithium, n-hexyllithium or lithium diisopropylamide (LDA), or a mixture of bases thereof, in an inert solvent such as tetrahydrofuran (THF), diethyl ether, pentane or hexane, or a mixture of solvents thereof, and reacting the resulting dianion with an ester of the formula (III)

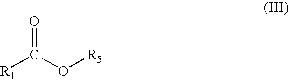

wherein R₁ has meaning as defined above, and R₅ is C₁-C₇-alkyl, preferably methyl, to afford compounds of the formula (IV)

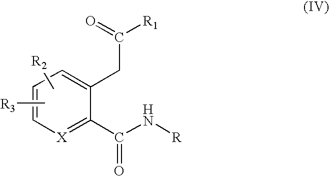

wherein R, R₁, R₂, R₃ and X have meanings as defined above. Preferably, the dianion is generated using a mixture of n-hexyllithium and LDA in THF at a temperature ranging from about −78° C. to about −30° C. More preferably, the temperature ranges from about −55° C. to about −35° C. Preferably, the molar ratio of n-hexyllithium to LDA initially present in the reaction mixture ranges from about 1:1 to about 1:1.5, and the initial molar ratio of the base to a compound of formula (II) ranges from about 2:1 to about 5:1. The subsequent exothermic condensation reaction with a compound of formula (III) is preferably conducted at an initial reaction temperature ranging from about −15° C. to about 10° C. More preferably, the initial temperature ranges from about −5° C. to 5° C. The molar ratio of a compound of formula (III) to a compound of formula (II) originally present in the reaction mixture may range from about 2:1 to about 1:1. Preferably, the molar ratio is about 1.3:1.

Compounds of formula (II) and (III) are known, or may be prepared according to methods well known in the art, or using methods described herein in the illustrative Examples, e.g., compounds of formula (II) wherein R is t-butyl may be obtained by reacting compounds of the formula (VIII)

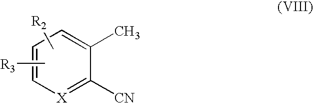

wherein R₂, R₃ and X have meanings as defined above, with isobutylene, or an equivalent thereof, e.g., t-butanol or t-butyl acetate, preferably t-butyl acetate, in the presence of an acid catalyst and an inert solvent. Accordingly, the above Ritter reaction may be conducted using concentrated sulfuric acid as the acid catalyst and acetic acid as the solvent at a temperature ranging from about 0° C. to about 50° C., preferably, at a temperature ranging from about 20° C. to about 30° C. Preferably, the initial molar ratio of the acid catalyst to a compound of formula (VIII) ranges from about 0.5:1 to about 5:1, and the initial molar ratio of isobutylene, or an equivalent thereof, to a compound of formula (VIII) ranges from about 1:1 to about 5:1. More preferably, the initial molar ratio of the acid catalyst to a compound of formula (VIII) is about 2.25:1, and the initial molar ratio of isobutylene, or an equivalent thereof, to a compound of formula (VIII) is about 2:1.

Compounds of formula (IV) may then be cyclized in the presence of an ammonium salt, e.g., ammonium acetate, and a suitable solvent such as acetic acid to obtain compounds of the formula (V)

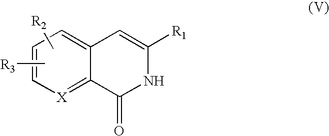

wherein R₁, R₂, R₃ and X have meanings as defined above. The cyclization may be carried out using an excess of an ammonium salt at a temperature ranging from room temperature (RT) to about 150° C. Preferably, the reaction is conducted at a temperature ranging from about 100° C. to about 115° C. The molar ratio of the ammonium salt to a compound of formula (IV) initially present in the reaction mixture may range from about 5:1 to about 20:1. Preferably, the molar ratio of the ammonium salt to a compound of formula (IV) is about 10:1.

Compounds of the formula (V) may then be treated with a halogenating agent such as phosphorus oxychloride, phosphorus pentachloride, phosphorus oxybromide or phosphorus pentabromide, preferably phosphorus oxychloride or phosphorus oxybromide, in an organic solvent such as acetonitrile, DCM or toluene, preferably toluene, to form compounds of the formula (VI)

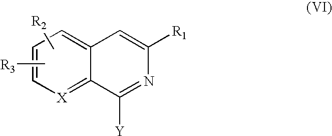

wherein R₁, R₂, R₃ and X have meanings as defined above, and Y is chloro or bromo. The reaction may be conducted in the presence of an excess of a halogenating agent at a temperature ranging from RT to about 150° C. Preferably, the reaction is conducted at a temperature ranging from about 100° C. to about 115° C. The molar ratio of the halogenating agent to a compound of formula (V) initially present in the reaction mixture may range from about 3:1 to about 15:1. Preferably, the molar ratio of the halogenating agent to a compound of formula (V) is about 10:1.

Finally, compounds of formula (VI) may be coupled in the presence of a catalyst, preferably a palladium catalyst, e.g., tetrakis(triphenylphosphine)palladium(0) or palladium(I)tri-t-butyl-phosphine bromide dimer, and a base such as sodium hydroxide (NaOH) or sodium or potassium carbonate in an appropriate solvent, e.g., water, acetonitrile, methanol, ethanol or THF, or a mixture of solvents thereof, with a compound of the formula (VII)

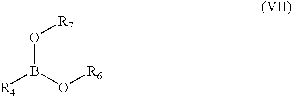

wherein R₄ has a meaning as defined for formula (I), and R₆ and R₇ are hydrogen or C₁-C₇-alkyl, or R₆ and R₇ combined are C₂-C₃ alkylene optionally substituted by one or two of C₁-C₄-alkyl that together with the boron and the oxygen atoms form a 5- or 6-membered ring, to afford compounds of formula (I) wherein R₁, R₂, R₃, R₄ and X have meanings as defined above. Preferably, R₆ and R₇ are hydrogen and the above Suzuki reaction is conducted in water at a temperature ranging from RT to about 100° C. More preferably, the reaction is conducted at a temperature ranging from about 80° C. to about 85° C. The molar ratio of a compound of formula (VII) to a compound of formula (VI) initially present in the reaction mixture may range from about 1:1 to about 2:1, preferably, the molar ratio is about 1.2:1. The molar ratio of the base to a compound of formula (VI) initially present in the reaction mixture may range from about 1:1 to about 5:1, preferably, the molar ratio is about 2.5:1. The molar ratio of the palladium catalyst to a compound of formula (VI) may range from about 0.001:1 to about 0.01:1, preferably, the molar ratio is about 0.004:1.

The present invention further includes any variant of the above process, in which an intermediate product obtainable at any stage thereof is used as starting material, e.g., compounds of formula (IV) and (V), and the remaining steps are carried out, or in which intermediates are converted into each other according to the methods of the present invention, or in which the reaction components are used in the form of their salts.

Preferably, compounds of formula (I) are prepared by a process of the present invention wherein R is t-butyl.

More preferably, compounds of formula (I) are prepared by a process of the present invention wherein R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; R₄ is phenyl optionally substituted by one, two, three or four substituents selected from C₁-C₇-alkyl, halo, hydroxy, C₁-C₇-alkoxy, C₁-C₇-alkylthio and nitro; R₅ is methyl; R₆ and R₇ are hydrogen; and X is N.

Most preferably, compounds of formula (I) are prepared by a process of the present invention wherein R₁ is 4-carboxycyclohexyl, and R₄ is 3-fluorophenyl.

In a particular embodiment, a process of the present invention is employed for the manufacture of a compound of formula (I) which is 4-[8-(3-fluorophenyl)-[1,7]-naphthyridin-6-yl-trans-cyclohexanecarboxylic acid.

Compounds of formula (IV), (V) and (VI) are useful as intermediates for the manufacture of compounds of formula (I). Compounds of formula (I) are inhibitors of PDE4 enzyme and, thus, may be employed for the treatment of chronic inflammatory diseases such as asthma, COPD and rheumatoid arthritis.

Preferred are compounds of formula (IV) wherein R is t-butyl, R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; and X is N. More preferred are compounds of formula (IV) wherein R₁ is 4-carboxycyclohexyl.

Preferred are compounds of formula (V) wherein R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; and X is N. More preferred are compounds of formula (V) wherein R₁ is 4-carboxycyclohexyl.

Preferred are compounds of formula (VI) wherein R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; Y is chloro; and X is N. More preferred are compounds of formula (VI) wherein R₁ is 4-carboxycyclohexyl.

The processes described herein above are preferably conducted under inert atmosphere, more preferably under nitrogen atmosphere.

When required, protecting groups may be introduced to protect the functional groups present from undesired reactions with reaction components under the conditions used for carrying out a particular chemical transformation of the present invention. The need and choice of protecting groups for a particular reaction is known to those skilled in the art and depends on the nature of the functional group to be protected (hydroxyl group, thiol etc.), the structure and stability of the molecule of which the substituent is a part and the reaction conditions.

Well-known protecting groups that meet these conditions and their introduction and removal are described, for example, in McOmie, “Protective Groups in Organic Chemistry”, Plenum Press, London, NY (1973); Greene and Wuts, “Protective Groups in Organic Synthesis”, John Wiley and Sons, Inc., NY (1999).

Compounds of the present invention may be isolated using conventional methods known in the art, e.g., extraction and filtration. Furthermore, such methods may be combined, e.g., with the use of solid phase scavengers to remove unreacted starting materials or reaction by-products. For example, as described herein in the illustrative Examples SMOPEX fibres may be employed in Suzuki coupling to remove palladium from the reaction mixture.

Depending on the choice of starting materials, compounds of formula (I), and intermediates thereof, may be in the form of one of the possible isomers, or mixtures thereof, e.g., as substantially pure geometric (e.g. cis and trans) isomers, optical isomers (antipodes), racemates, or mixtures thereof. The aforesaid possible isomers, or mixtures thereof, are all within the purview of the invention.

Any resulting mixtures of isomers may be separated on the basis of their different physico-chemical properties into the pure, e.g., geometric, isomers by conventional methods such as chromatography and/or crystallization, preferably crystallization. For example, compounds of formula (I), in particular, 4-[8-(3-fluorophenyl)-[1,7]-naphthyridin-6-yl-trans-cyclohexane-carboxylic acid may be obtained in high geometric purity by crystallization from a mixture of acetonitrile and water followed by recrystallization from a mixture of ethanol and water as described herein in the illustrative Examples.

Any resulting racemates of final products, or intermediates thereof, can be resolved into the optical antipodes by known methods, e.g., by separation of the diastereoisomeric salts thereof, obtained with an optically active acid or base, and later liberating the optically active acidic or basic parent compound. Racemic products may also be resolved employing chiral chromatography, e.g., high pressure liquid chromatography (HPLC) using a chiral adsorbent.

The following Examples are intended to illustrate the invention and are not to be construed as being limitations thereon. If not mentioned otherwise, all evaporations are performed under reduced pressure, preferably between about 7.5 and 112.5 mm Hg (=10-150 mbar). The structure of final products, intermediates and starting materials is confirmed by standard analytical methods, e.g., microanalysis, melting point (mp) and spectroscopic characteristics (e.g., MS, IR, NMR). Abbreviations used are those conventional in the art.

In the case of geometric isomers, e.g. cis and trans isomers, the following HPLC method may also be used to identify compounds of the present invention by their retention times: DYNAMAX Model SD-200 on symmetry Column (C18, 5 μm, 250 mm×4.6 mm, Waters); flow rate of 1.0 mL/min; and using a mixture of water with 0.05% of trifluoroacetic acid (TFA, v/v) and acetonitrile with 0.05% of TFA (v/v) as the eluent with gradient from 90/10 to 10/90 and UV detection at wavelength of 210 nm; or alternatively using a pH 3 buffer solution in acetonitrile as the eluent.

### EXAMPLE 1

### 3-Methyl-pyridine-2-carboxylic acid t-butylamide

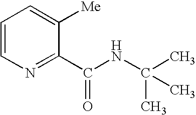

A one liter 4-necked LabMax (equipped with mechanical stirrer and 250-mL graduated addition funnel and nitrogen bubbler) is charged with 2-cyano-3-methylpyridine (0.8 mol, 94.4 g) and acetic acid (2.62 mol, 150.0 mL). The white suspension is stirred at RT at a rate of 250 rpm. Concentrated sulfuric acid (1.8 mol, 96.0 mL) is added over 0.5 h to the reaction mixture keeping the temperature below 30° C. with cooling. During the addition, the solution is first an opaque, white solution and then becomes clear and colorless by the end of the addition. t-Butyl acetate (1.6 mol, 215.6 mL) is added dropwise over 45 minutes keeping the reaction under a constant and gentle N₂ stream and the temperature at 25±4° C. After addition, the resulting clear colorless solution is mechanically stirred at RT for 4 hours. The reaction mixture is then held at RT for another 8 hours to guarantee complete reaction. The reaction is quenched by dropwise addition into a 5-L round-bottom flask containing a mechanically-stirred 9.0% aqueous NaOH solution (ice-cooled to 8±4° C., 360 g of NaOH in 3.64 kg of water) over 40 minutes. By the end of the addition, the solution temperature rises to 27° C., and a significant amount of solid is observed. The mixture is stirred at RT for 1.5 hours further, the reaction vessel is drained while stirring and the solid is collected by filtration. The collected solids are suspended in water (600 g) and stirred for 0.5 hours, then collected by filtration and dried under vacuum (44±5° C., 25 mbar) for 14 hours to afford 3-methyl-pyridine-2-carboxylic acid t-butylamide as a white crystalline solid.

### EXAMPLE 2

### 1,4-Cyclohexanedicarboxylic acid monomethyl ester

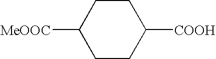

A five liter 4-necked round flask (equipped with mechanical stirrer, nitrogen inlet, condenser and digital temperature controller/probe) is charged with 1,4-cyclohexane-dicarboxylic acid dimethyl ester (4.792 mol, 1.01 kg), and the funnel is rinsed once with methanol (79 g, 100 mL). The homogenous solution is cooled at 16±3° C. over 15 minutes. A warm solution (47±3° C.) of potassium hydroxide (2.396 mol, 158.2 g) in methanol (1.343 kg, 1.70 L) is added at 16° C. to 19° C. over 1 hour. The addition funnel is rinsed once with methanol (158 g, 200 mL). The pale yellow homogenous mixture (pH˜14) is warmed slowly to 65° C. over 1.5 hours, then refluxed at 65±3° C. for 2 hours (pH˜8.5). The reaction mixture is cooled to 35±3° C. The contents are concentrated at 35±3° C. (15-150 mbar) to give a hazy viscous oil which is flushed once with heptane (240 g, 350 mL) at 38±3° C. (15-150 mbar) to afford a white stirrable paste. Water (2.50 kg) and heptane (686 g, 1.0 L) are added and the mixture is stirred at 22±3° C. for 15 minutes to give two clear layers (pH˜8.5). A solution of potassium carbonate (20 g) in water (100 g) is then added and the mixture is stirred for 15 minutes to adjust pH of the solution to 10.5. The layers are allowed to settle for 15 minutes, then separated. The organic layer is washed once with water (100 g), and the previously separated aqueous layer and water wash are combined. This aqueous solution is extracted once with heptane (686 g, 1.0 L) and the layers are separated. The organic layer is washed once with water (100 g), and the previously separated aqueous layer and the water wash are again combined (volume ˜3.3 L). Sodium chloride (250 g) is added and the mixture is stirred at 22±3° C. for 15 minutes, then the aqueous solution is transferred into a 12-L separatory flask. Methyl-t-butyl ether (MTBE, 2.34 kg, 3.16 L) and a solution of concentrated hydrochloric acid (HCl, 37 wt %, 209 g) in water (174 mL) are added into the mixture at 22±3° C. to adjust the pH to 5.50±0.1 (total volume ˜6.5 L). The aqueous layer is separated and the organic layer is washed once with water (100 g). The layers are allowed to settle for 3 hours or overnight (possible hold point), then separated. The organic solution is transferred into a 5-L 4-necked round flask (equipped with mechanical stirrer, nitrogen inlet, condenser and digital temperature controller/probe), then heated to 50±3° C. over 30 minutes and MTBE is distilled off at 50° C. to 71° C. (reactor temperature) under atmospheric pressure to afford a viscous oil (˜300 mL volume). Heptane (997 g) is added over 15 to 30 minutes under an efficient agitation (400 rpm) and the pot temperature is maintained at 60±3° C. The hazy contents are cooled slowly to about 56° C. and the suspension is maintained at 54±3° C. for 1 hour. The slurry is cooled slowly to 9±3° C. over 1.5 hours and maintained at this temperature for 30 minutes. The solids are collected by filtration through a polypropylene filter pad and Büchner funnel at 9±3° C., then the flask and filter cake are washed with the original filtrate (9±3° C.). The cake is air-dried for 1 hour (˜150 mbar), then dried in a vacuum oven (60±3° C., 15 mbar) for 18 hours to give 1,4-cyclohexanedicarboxylic acid monomethyl ester as a white solid: mp 85-87° C.

### EXAMPLE 3

### 4-[2-(2-t-Butylcarbamoyl-pyridin-3-yl )-acetyl]-cyclohexanecarboxylic acid

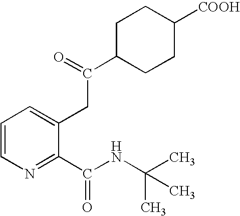

A five liter 4-necked flask (equipped with mechanical stirrer, gas outlet, gas inlet, and thermocouple, addition funnel) is charged with THF (1.9 L) and diisopropylamine (1.25 mol, 126.5 g). The solution is cooled to about −40° C. to −50° C. A solution of n-hexyllithium in hexane (4.54 mol, 645 g) is added slowly (30 to 40 minutes) and the mixture is stirred for 30 minutes at this temperature. A solution of 3-methyl-pyridine-2-carboxylic acid t-butylamide from Example 1 (0.5 mol, 96 g) in THF (300 mL) is added while maintaining the temperature at about −40° C. to −50° C. (30 minutes). The reaction is stirred for another 30 minutes and then warmed to about 0° C. to 3° C. A solution of cyclohexane-1,4-dicarboxylic acid monomethyl ester from Example 2 (0.644 mol, 120 g) in THF (300 mL) is added as fast as possible (7 to 10 minutes). During the addition, the internal temperature rises from about 3° C. to about 36° C. Vigorous stirring is necessary as solids tend to separate at this stage. The reaction is stirred at this temperature for 1.5 hours, then cooled to about −5° C. to −20° C. Water (1.25 L) is added slowly and the mixture is warmed to about 10° C. to 20° C. The layers are separated and the aqueous layer is extracted with t-butyl methyl ether (500 mL) and the aqueous solution is held at about 20° C. to 2° C. for at least 12 hours. 6 N aqueous HCl (365 mL) is added at 10±3° C. to adjust the pH to about 5.8±0.2. The mixture is stirred at this pH for 30 minutes until solid formation is observed. 6 N aqueous HCl is added slowly to reach a pH of about 5.0. The suspension is stirred at about 0° C. to 5° C. for 1 hour and the solids are collected by filtration using Buchner funnel and filter cloth. The solids are washed with water (300 mL) and dried in the oven at 50° C. (25 mbar) for 14 hours to give 4-[2-(2-t-butylcarbamoyl-pyridin-3-yl)-acetyl]-cyclohexane-carboxylic acid as an off-white powder and about a 85:15 mixture of the trans and cis isomers: mp ˜160° C.; MS 347.1 [M+1]⁺.

### EXAMPLE 4

### 4-(8-Oxo-7,8-dihydro-[1,7]naphthyridin-6-yl)-cyclohexanecarboxylic acid

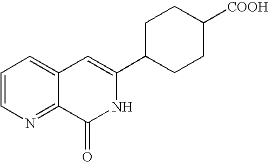

A three liter 4-necked round-bottomed flask (equipped with mechanical stirrer and a reflux condenser) is charged with 4-[2-(2-t-butylcarbamoyl-pyridin-3-yl)-acetyl]-cyclohexane-carboxylic acid from Example 3 (0.393 mol, 0.136 kg), ammonium acetate (3.93 mol, 303 g), and acetic acid (275 g). The white suspension is stirred at RT at a rate of 250 rpm for 10 minutes until the reaction becomes a thick homogeneous slurry. The reaction is heated to 108±3° C. over 40 minutes, and the resulting clear, dark-amber reaction mixture is stirred at this temperature for 12 hours further. The mixture is cooled to 50° C. and water (1.5 L) is added and the mixture is cooed further to about 10° C. After 1.5 hours, reaction vessel is drained and the precipitated solids are collected by filtration. The collected solids are washed with a chilled (10±5° C.) mixture of water (600 mL) and methanol (76 mL), then dried under vacuum (60±5° C., 25 mbar) for 14 h to afford 4-(8-oxo-7,8-dihydro-[1,7]naphthyridin-6-yl)-cyclohexanecarboxylic acid as an off-white powder and about a 93:7 mixture of the trans and cis isomers: mp>270° C.; MS 273.3 [M+1]⁺.

### EXAMPLE 5

### 4-(8-Chloro-[1,7]naphthyridin-6-yl)-cyclohexanecarboxylic acid

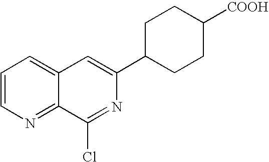

A two liter 4-necked round-bottomed flask (equipped with mechanical stirrer, nitrogen inlet, condenser and digital temperature controller/probe) is charged with 4-(8-oxo-7,8-dihydro-[1,7]naphthyridin-6-yl)-cyclohexanecarboxylic acid from Example 4 (0.257 mol, 70.9 g), toluene (770 mL), and phosphorus oxychloride (2.671 mol, 247 mL). The suspension is heated slowly to about 106° C. over 1 hour, then refluxed gently at 108±3° C. for 6.5 hours to give a dark homogenous mixture. The reaction is cooled to 20±3° C. over 30 minutes, and then poured slowly into cold (about 2° C.) water (3.03 L) in a 5-L 4-necked round-bottomed flask. The temperature is maintained at 5±3° C. for 1 hour. The two liter flask is rinsed once with toluene (350 mL) and the rinse solution is combined with the cooled reaction mixture. The combined mixture is stirred at 5±3° C. for 1.5 hours. A solution of sodium hydroxide (413 g) in water (413 mL) is added over 30 to 60 minutes while maintaining the reaction temperature at 5±3° C. to adjust the pH of the mixture to 3.1±0.2 (end volume ˜4.7 L). The suspension is warmed to 7±3° C. over 10 minutes, and the solids are collected by filtration through a polypropylene filter cloth and Büchner funnel, then washed twice with water (2×250 mL). The solids are air-dried for 1 hour at 200 mbar, then dried in a vacuum oven (50±3° C., 15 mbar) for 18 hours to give 4-(8-chloro-[1,7]naphthyridin-6-yl)-cyclo-hexanecarboxylic acid as a tan solid and about a 81:19 mixture of the trans and cis isomers: mp 213-214° C. (with decomposition); MS 291.08 [M+1]⁺.

### EXAMPLE 6

### 4-[8-(3-Fluoro-phenyl )-[1,7]naphthyridin-6-yl]-cyclohexanecarboxylic acid

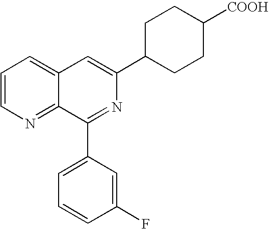

A 500 mL 4-necked flask (equipped with mechanical stirrer, gas outlet, gas inlet, thermocouple and condenser) is charged with water (400 mL), potassium carbonate (0.499 mol, 69 g), 4-(8-chloro-[1,7]naphthyridin-6-yl)-cyclohexanecarboxylic acid from Example 5 (0.2 mol, 58.2 g), 3-fluorophenylbronic acid (0.24 mol, 33.6 g) and palladium(I)tri-t-butylphosphine bromide dimer (0.809 mmol, 629 mg). The resulting solution is heated to 83±3° C., and maintained at this temperature for 2 hours. The reaction is monitored by HPLC. After the completion of the reaction, water (400 mL) is added, and the reaction mixture is extracted with MBTE (3×240 mL). HCl (700 mL, 37 wt %) is added to the aqueous phase at 10° C. to 30° C. followed by addition of SMOPEX 110 (7.0 g), and the mixture is heated at 60° C. for 1 hour. The hot solution is filtered through a column packed with Celite™ filter material and activated carbon. The column is washed with hot solution (40° C. to 50° C.) of aqueous HCl (6 N, 422.4 g), and the filtrate is neutralized with aqueous NaOH (727.2 g, 50%) to pH 9 at <20° C. The mixture is stirred at this temperature for 3 hours, then adjusted to pH of about 2 to 3 by adding aqueous HCl (6 N, 37.0 g) and stirring is continued for 3 hours at about 0° C. to 5° C. The solids are collected by filtration, washed with water (200 mL) and dried at 60° C. for 14 hours to give 4-[8-(3-fluoro-phenyl)-[1,7]naphthyridin-6-yl]-cyclohexanecarboxylic acid as a light yellow solid and about a 82:18 mixture of the trans and cis isomers: MS 351.16 [M+1]⁺.

### EXAMPLE 7

### 4-[8-(3-Fluoro-phenyl)- [1,7]naphthyridin-6-yl]-trans-cyclohexanecarboxylic acid

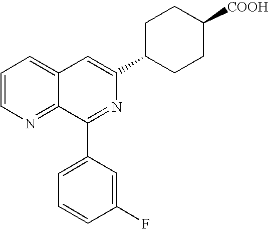

A one liter 4-necked flask (equipped with mechanical stirrer, gas outlet, gas inlet, and thermocouple, condenser and addition funnel) is charged with 4-[8-(3-fluoro-phenyl)-[1,7]naphthyridin-6-yl]-cyclohexanecarboxylic acid from Example 6 (0.217 mol, 76.0 g), acetonitrile (660 mL), water (53 mL). The mixture is heated to about 30° C. to 40° C. and adjusted to a pH of 2.0±0.5 by addition of aqueous NaOH (2 N, 18 mL). If the volume of NaOH is less than 18 mL (2 N), water is added to adjust the ratio of acetonitrile to aqueous NaOH to about 10 to 1 v/v. SMOPEX 110 (7.6 g) is added and the mixture is heated at about 70° C. for 4 hours. The hot solution is filtered and rinsed with hot acetonitrile (50 mL). The filtrate is seeded with 4-[8-(3-fluoro-phenyl)-[1,7]naphthyridin-6-yl]-cyclohexane-carboxylic acid from Example 5 and then stirred at 18±3° C. for 4 hours. The solids are collected by filtration, washed with water (110 mL) and dried in the oven at 50° C. for 14 hours to give 4-[8-(3-fluoro-phenyl)-[1,7]naphthyridin-6-yl]-trans-cyclohexanecarboxylic acid (trans >98%) as a white solid.

### EXAMPLE 8

### 4-[8-(3-Fluoro-phenyl)-[1,7]naphthyridin-6-yl]-trans-cyclohexanecarboxylic acid

A one liter 4-necked flask (equipped with mechanical stirrer, gas outlet, gas inlet, and thermocouple, condenser, and addition funnel) is charged with 4-[8-(3-fluoro-phenyl)-[1,7]naphthyridin-6-yl]-cyclohexanecarboxylic acid from Example 7 (0.097 mol, 34.0 g), SMOPEX 110 (3.4 g), ethanol (268.6 g) and the mixture is heat to 70° C. After 3 hours at this temperature, the hot solution is filtered and the reactor is rinsed with ethanol (39.5 g). If solids are formed during holding before transferring, the filtrate is heated to 60° C. to dissolve the solids. The filtrate is transferred to another reactor maintaining the temperature above 50° C. The transferring line is rinsed with ethanol (39.5 g) and the solution is heated to about 60° C. Water (440 g) is added slowly (on this scale the addition time is 30 minutes) while maintaining the temperature at 55±5° C. Solids are formed during the addition. The temperature is maintained at 50° C. for another 30 minutes after addition. The mixture is cooled to 13±3° C. over 2 hours and held at this temperature for 2 hours further. The solids are collected by filtration, washed with pre-cold (about 10° C. to 15° C.) ethanol/water (25 mL/25 mL) and dried in oven at 50° C. for 14 hours to give 4-[8-(3-fluoro-phenyl)-[1,7]naphthyridin-6-yl]-trans-cyclohexanecarboxylic acid (trans >99%) as a white solid.

1. A process for the preparation of compounds of formula (I) 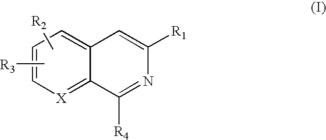
wherein R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,
or R₁ is C₃-C₁₂-cycloalkyl optionally substituted by one or two of C₁-C₇-alkyl, hydroxy, C₁-C₇-alkoxy, C₁-C₇-alkylthio or carboxy,
or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₁ is heteroaryl optionally substituted by C₁-C₇- alkyl, C₁-C₇-alkoxy or halo;
R₂ and R₃ are independently hydrogen or C₁-C₂₀-alkoxy;
R₄ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₄ is heteroaryl optionally substituted by C₁-C₇- alkyl, C₁-C₇-alkoxy or halo; and
X is N or CH;
or a salt thereof;
which process comprises coupling compounds of formula (VI) 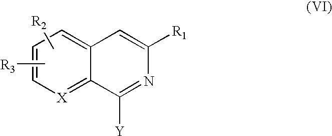
wherein R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,
or R₁ is C₃-C₁₂-cycloalkyl optionally substituted by one or two of C₁-C₇-alkyl, hydroxy, C₁-C₇-alkoxy, C₁-C₇-alkylthio or carboxy,
or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₁ is heteroaryl optionally substituted by C₁-C₇-alkyl, C₁-C₇-alkoxy or halo;
R₂ and R₃ are independently hydrogen or C₁-C₂₀-alkoxy;
X is N or CH;
and Y is chloro or bromo in the presence of a catalyst and a base with a compound of the formula (VII) 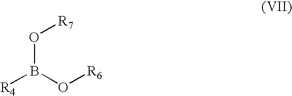
wherein R₄ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₄ is heteroaryl optionally substituted by C₁-C₇-alkyl, C₁-C₇-alkoxy or halo;
and R₆ and R₇ are hydrogen or C₁-C₇-alkyl,
or R₆ and R₇ combined are C₂-C₃ alkylene optionally substituted by one or two of C₁-C₄-alkyl that together with the boron and the oxygen atoms form a 5- or 6-membered ring.

2. A process according to claim 1, wherein R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; R₄ is phenyl optionally substituted by one, two, three or four substituents selected from C₁-C₇-alkyl, halo, hydroxy, C₁-C₇-alkoxy, C₁-C₇-alkylthio and nitro; R₅ is methyl; R₆ and R₇ are hydrogen; Y is chloro; and X is N.

3. A process according to claim 1 or 2 wherein R₁ is C₃-C₆-cycloalkyl substituted by carboxy and R₄ is phenyl substituted by halo.

4. A process according to claim 3, wherein R₁ is 4-carboxycyclohexyl; and R₄is 3-fluorophenyl.

5. A process according to any preceding claim, wherein a compound of formula (I) is 4-[8-(3-fluorophenyl)-[1,7]-naphthyridin-6-yl-trans-cyclohexanecarboxylic acid.

6. A process according to claim 1, wherein compounds of formula (VI) as defined in claim 1 are prepared by the steps of:
(a) treating compounds of the formula (II) 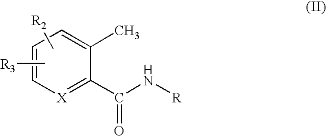
wherein R is C₁-C₇-alkyl; and R₂, R₃ and X are as defined in claim 1; with a base in an inert solvent; and reacting the resulting dianion with an ester of the formula (II) 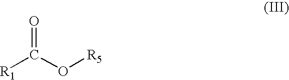
wherein R₁ is as defined in claim 1; and R₅ is C₁-C₇-alkyl; to afford compounds of the formula (IV) 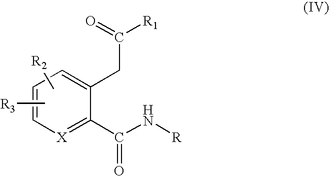
wherein R, R₁, R₂, R₃ and X are as defined in claim 1;
(b) cyclizing compounds of formula (IV) in the presence of an ammonium salt and a suitable solvent to obtain compounds of the formula (V) 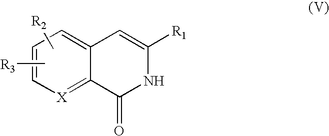
wherein R₁, R₂, R₃ and X are as defined in claim 1; and
(c) treating compounds of the formula (V) with a halogenating agent in an organic solvent to form compounds of the formula (VI) 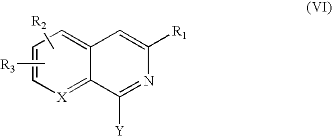
wherein R₁, R₂, R₃ and X are as defined in claim 1; and Y is chloro or bromo.

7. A process according to claim 6, wherein R is t-butyl.

8. A process according to claim 6 or 7, wherein R₁ is C₃-C₁₂-cycloalkyl optionally substituted by one or two of C₁-C₇-alkyl, hydroxy, C₁-C₇-alkoxy, C₁-C₇-alkylthio or carboxy; R₂ and R₃ are hydrogen; R₄ is phenyl optionally substituted by one, two, three or four substituents selected from C₁-C₇-alkyl, halo, hydroxy, C₁-C₇-alkoxy, C₁-C₇-alkylthio and nitro; R₅ is methyl; R₆ and R₇ are hydrogen; Y is chloro; and X is N.

9. A process according to any one of claims 6 to 8, wherein R₁ is C₃-C₆-cycloalkyl substituted by carboxy and R₄ is phenyl substituted by halo.

10. A process according to claim 9, wherein R₁ is 4-carboxycyclohexyl; and R₄ is 3-fluorophenyl.

11. A process according to any one of claims 6 to 10, wherein a compound of formula (I) is 4-[8-(3-fluorophenyl)-[1,7]-naphthyridin-6-yl-trans-cyclohexanecarboxylic acid.

12. A compound of the formula (IV) 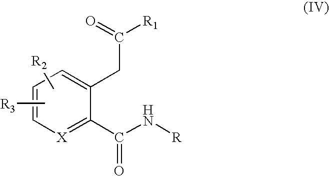
or a salt thereof, wherein
R is C₁-C₇-alkyl;
R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,
or R₁ is C₃-C₁₂-cycloalkyl optionally substituted by one or two of C₁-C₇-alkyl, hydroxy, C₁-C₇-alkoxy, C₁-C₇-alkylthio or carboxy,
or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₁ is heteroaryl optionally substituted by C₁-C₇-alkyl, C₁-C₇-alkoxy or halo;
R₂ and R₃ are independently hydrogen or C₁-C₂₀-alkoxy; and
X is N.

13. A compound of the formula (V) 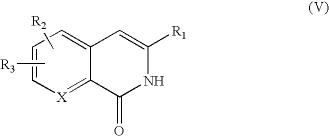
or a salt thereof, wherein
R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,
or R₁ is C₃-C₁₂-cycloalkyl optionally substituted by one or two of C₁-C₇-alkyl, hydroxy, C₁-C₇-alkoxy, C₁-C₇-alkylthio or carboxy,
or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₁ is heteroaryl optionally substituted by C₁-C₇-alkyl, C₁-C₇-alkoxy or halo, provided that R₁ is not phenyl, 4-methoxyphenyl or 3-methyl-2-pyridinyl;
R₂ and R₃ are independently hydrogen or C₁-C₂₀-alkoxy; and
X is N.

14. A compound of the formula (VI) 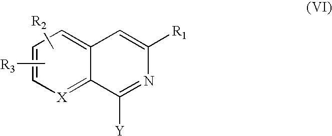
or a salt thereof, wherein
R₁ is C₁-C₂₀-alkyl optionally substituted by one or two of hydroxy, C₃-C₁₂-cycloalkyl, C₆-C₁₂-aryl, C₁-C₇-alkoxy, thiol, C₁-C₇-alkylthio or carboxy,
or R₁ is C₃-C₁₂-cycloalkyl optionally substituted by one or two of C₁-C₇-alkyl, hydroxy, C₁-C₇-alkoxy, C₁-C₇-alkylthio or carboxy,
or R₁ is C₆-C₁₂-aryl optionally substituted by one, two, three or four substituents selected from C₁-C₇-alkyl, halo, hydroxy, C₁-C₇-alkoxy, C₁-C₇-alkylthio and nitro,
or R₁ is heteroaryl optionally substituted by C₁-C₇-alkyl, C₁-C₇-alkoxy or halo,
provided that (a) R₁ is not 4-carboxyphenyl when Y is bromo, or (b) R₁ is not 3-methyl-2-pyridinyl when Y is chloro;
R₂ and R₃ are independently hydrogen or C₁-C₂₀-alkoxy;
X is N; and
Y is chloro or bromo.
